(19) United States
(12) Patent Application Publication
(10) Pub. No.: US 2025/0213917 A1
(43) Pub. Date: Jul. 3, 2025

(54) DEVICES, SYSTEMS, AND METHODS FOR PELVIC FLOOR PHYSICAL THERAPY

(71) Applicant: The University of North Carolina at Chapel Hill, Chapel Hill, NC (US)

(72) Inventors: Erin Teeter Carey, Chapel Hill, NC (US); Devin Kerry Hubbard, Chapel Hill, NC (US); Nicole Lewis Wiley, Pittsboro, NC (US); Jennifer Rachel Harrington, Chapel Hill, NC (US)

(21) Appl. No.: 18/848,616

(22) PCT Filed: Mar. 23, 2023
(86) PCT No.: PCT/US2023/016013

§ 371 Date: Sep. 19, 2024

Related U.S. Application Data
(60) Provisional application No. 63/322,886, filed on Mar. 23, 2022.

(51) Int. Cl. A63B 23/20; A63B 21/00; A63B 71/06

(52) U.S. Cl. CPC A63B 23/20; A63B 21/00196; A63B 71/0622; A63B 2220/10

(57) ABSTRACT

The present subject matter relates to devices, systems, and methods for use in physical therapy of the pelvic floor and/or for treating pelvic floor dysfunction (PFD) and pelvic pain. The devices, systems, and methods can provide a pelvic floor physical therapy device that includes a handle portion configured to be gripped by a user; a body portion extending from the handle portion; and one or more sensors integrated within the body portion. The one or more sensors can be configured to collect sensor data regarding a position of the pelvic floor physical therapy device with respect to a pelvic floor location or pelvic floor muscle of a user. Visual or auditory feedback can be provided to the user regarding the position of the pelvic floor physical therapy device with respect to the pelvic floor location or pelvic floor muscle to guide a physical therapy treatment session.

### PRIORITY CLAIM

The present application claims the benefit of U.S. Provisional Patent Application Ser. No. 63/322,886, filed Mar. 23, 2023, the disclosure of which is incorporated herein by reference in its entirety.

### GOVERNMENT LICENSE RIGHTS

This invention was made with government support under Grant Number TR002489 awarded by the National Institutes of Health. The government has certain rights in the invention.

### TECHNICAL FIELD

The subject matter disclosed herein relates generally to devices, systems, and methods for use in physical therapy treatments. More particularly, the subject matter disclosed herein relates to devices, systems, and methods for use in physical therapy treatments for pelvic floor dysfunction and pelvic pain.

### BACKGROUND

Pelvic floor dysfunction (PFD) occurs when the pelvic floor muscles become too tight and/or too weak and fail to function properly. In particular, high tone pelvic floor disorder (HTPFD) is a chronic pelvic floor disorder characterized by tight, weakened, and/or painful pelvic floor muscles. Tension within the muscle directly affects its physiologic action, preventing appropriate coordination, contraction and relaxation. This may result in a wide range of genitourinary complaints, including discomfort, lower urinary tract symptoms, defecatory dysfunction, and sexual pain (dyspareunia). HTPFD can affect male and female patients and may experience chronic pain, voiding or defecatory dysfunction. Increased pelvic floor tone may underlie many urinary, gastrointestinal, and sexual complaints, even in the absence of pain. The etiology of HTPFD is broad, ranging from idiopathic (potentially caused by poor toileting habits) or triggered by visceral dysfunction (e.g. endometriosis or interstitial cystitis/bladder pain syndrome (IC/BPS), or musculoskeletal injuries, such as sacroiliac joint dysfunction or hip osteoarthritis and chronic low back pain). Clinical identification of high pelvic floor tone is subjective and provider-dependent.

Pelvic floor physical therapy (PFPT) is considered first-line treatment for the treatment of HTPFD. However, existing pelvic floor physical therapy (PT) entails in person therapy one to two times weekly for about 8 to 12 weeks, and barriers to therapy have been well described, including but not limited to financial limitations, perceived lack of utility, personal time constraints, difficulty with travel to PT, and fear of the intimacy of internal treatment.

Pelvic floor PT often includes the use of one or more dilator devices or myofascial release systems for patients with HTPFD. However, currently available dilators are static/mechanical without feedback, and can therefore be ineffective and potentially harmful, particularly when used by patients for self-therapy.

### SUMMARY

In accordance with this disclosure, devices, systems, and methods for use in physical therapy of the pelvic floor and/or for treating pelvic floor dysfunction (PFD) and pelvic pain are provided. In one aspect, a pelvic floor physical therapy device includes a handle portion configured to be gripped by a user; a body portion extending from the handle portion; and one or more sensors integrated within the body portion. The one or more sensors can be configured to collect sensor data regarding a position of the pelvic floor physical therapy device with respect to a pelvic floor location or pelvic floor muscle of a user.

In another aspect, a system for guided pelvic floor physical therapy includes a pelvic floor physical therapy device and a computer device comprising a display, a wireless data receiver, and a non-transitory computer readable medium programmed with a software application comprising executable instructions. The pelvic floor physical therapy device can include a handle portion configured to be gripped by a user; a body portion extending from the handle portion; one or more sensors integrated within the body portion; and a wireless data transmitter. The software application can include executable instructions that include receiving, from the pelvic floor physical therapy device, data from the one or more sensors regarding a position of the pelvic floor physical therapy device with respect to a pelvic floor location or pelvic floor muscle; comparing the data to a predetermined model of the pelvic floor location or pelvic floor muscle; and providing visual or auditory feedback regarding the position of the pelvic floor physical therapy device with respect to the pelvic floor location or pelvic floor muscle to guide a physical therapy treatment session.

In another aspect, a method of treating a subject is provided. The method can include positioning a pelvic floor physical therapy device with respect to a pelvic floor location or pelvic floor muscle of a user; receiving, from the pelvic floor physical therapy device, data from one or more sensors regarding the position of the pelvic floor physical therapy device with respect to the pelvic floor location or pelvic floor muscle; comparing the data to a predetermined model of the pelvic floor location or pelvic floor muscle; and providing visual or auditory feedback regarding the position of the pelvic floor physical therapy device with respect to the pelvic floor location or pelvic floor muscle to guide a physical therapy treatment session.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIGS. 1A and 1B are a side view and a perspective side view, respectively, of a pelvic floor physical therapy device according to an embodiment of the presently disclosed subject matter.

FIGS. 1C through 1F are perspective side views of a pelvic floor physical therapy device according to additional embodiments of the presently disclosed subject matter.

FIG. 2A is a side cutaway view of a pelvic floor physical therapy device according to an embodiment of the presently disclosed subject matter.

FIG. 2B is a perspective side view of internal components of the pelvic floor physical therapy device shown in FIG. 2A.

FIGS. 3 and 4 are perspective side views of internal components of a body portion of a pelvic floor physical therapy device according to different embodiments of the presently disclosed subject matter.

FIG. 5 is a perspective side view of internal components of a handle portion of a pelvic floor physical therapy device according to an embodiment of the presently disclosed subject matter.

FIG. 6 is a schematic diagram of a networked system for guiding pelvic floor physical therapy according to an embodiment of the presently disclosed subject matter.

FIG. 7 is a diagram showing a representative application of a pelvic floor physical therapy system for guided muscle stimulation according to an embodiment of the presently disclosed subject matter.

### DETAILED DESCRIPTION

The presently disclosed subject matter provides devices, systems, and methods for use in physical therapy of the pelvic floor and/or for treating pelvic floor dysfunction (PFD) and pelvic pain. The presently disclosed subject matter addresses some of the treatment gaps that are present when working with the traditional, mechanical wand that is usually used to address PFD. Conventional therapy for PFD often includes myofascial release, in which patients and/or physical therapists (or other medical professionals) use a purely mechanical wand, inserted into their vagina, to massage areas of tension known as trigger or tender points. This process can be extremely subjective, however, and patients can struggle to replicate the exercises at home without the live guidance from their therapists.

To address these issues, in one aspect, a device comprising an ergonomic manual wand, which can be used to provide sensory-based, interactive, pelvic floor physical therapy. In some embodiments, the device can further be used to provide guided, targeted trigger/tender point release and vibrational therapy on targeted muscles. More particularly, the disclosed devices can be designed to specifically target the superficial transverse perineal muscle, the pubococcygeus and puborectalis muscles of the levator ani complex, and/or the obturator internus muscles. Once inserted the device is calibrated to the vaginal introitus and superficial transverse perineal muscle of the patient upon which the device is to be used. In other embodiments, the device can be configured for rectal use. The disclosed device is referred to herein as a vaginal wand, rectal wand, wand device, smart wand, or simply wand.

In some embodiments, the device is a “smart” device that provides real-time sensing and user feedback for optimal use and effectiveness. For example, the device can be configured and/or calibrated to provide feedback to the user with respect to the amount of pressure to apply to release muscle tension. As a result, the disclosed wands and devices can be used, as described herein, for targeted treatment of pelvic floor dysfunction (PFD) and related conditions by application of the device within the vagina of a particular subject and directly in contact with the pelvic floor of that subject. In some embodiments, the presently disclosed devices can include vibration/massage components that are configured to help release trigger/tender points in the pelvic floor either directed by PT programming or self-directed by the user. The same or similar device could also be used for rectal applications. The disclosed devices, systems, and methods can be an important part of the home exercise program for pelvic floor dysfunction.

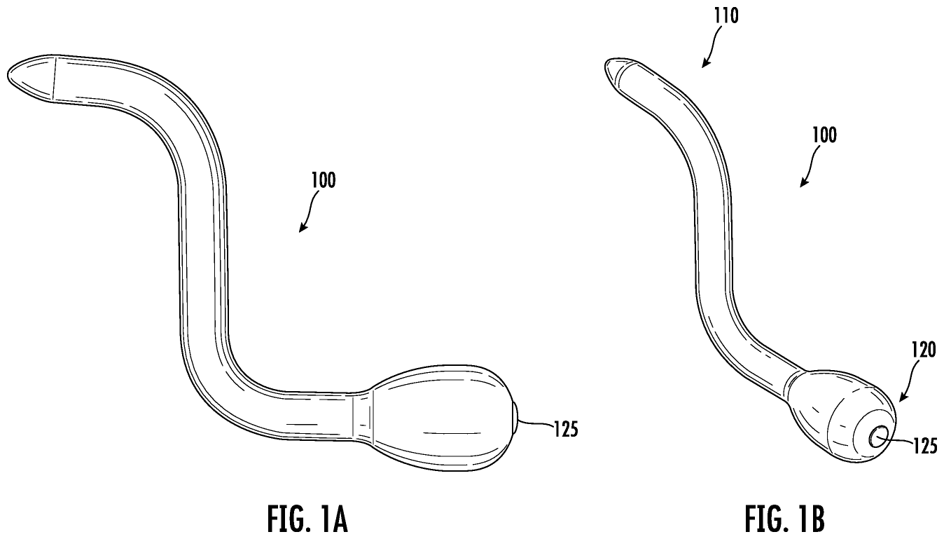

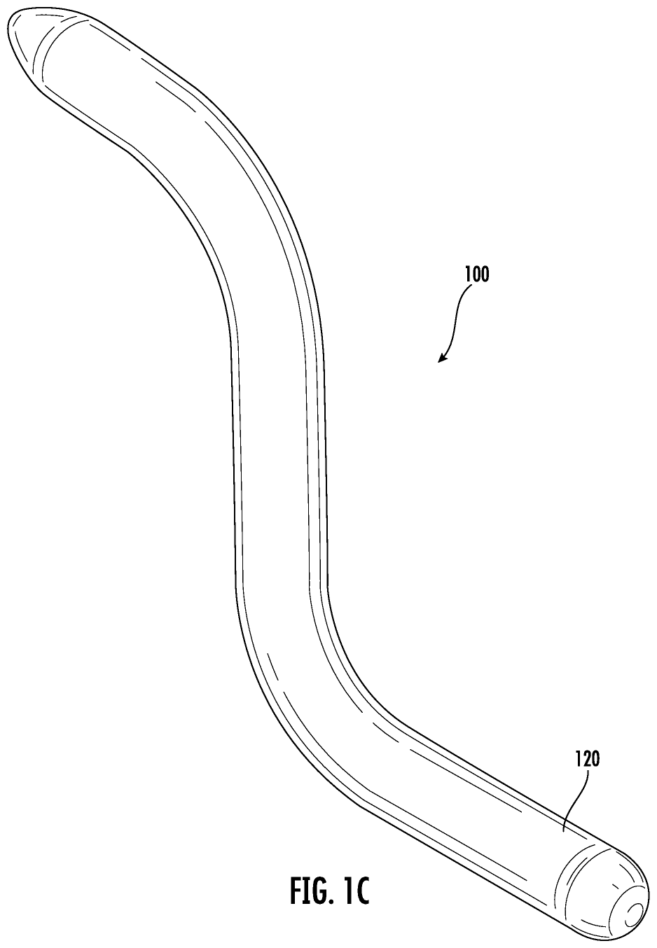

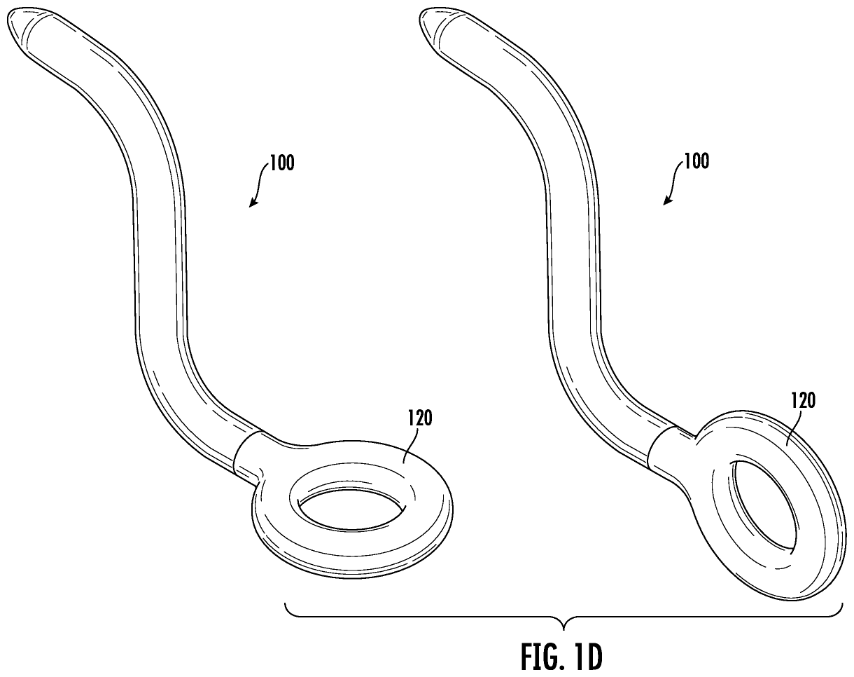

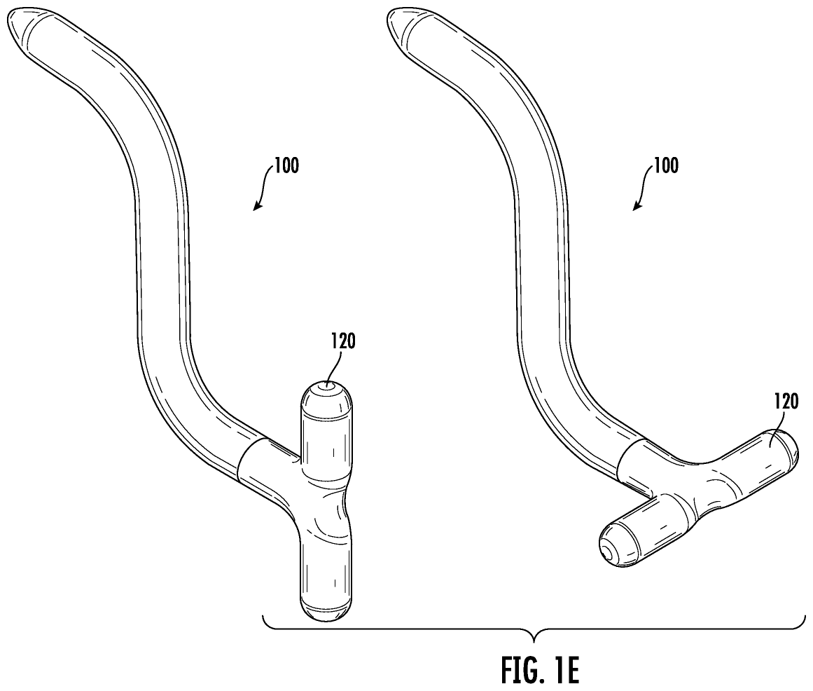

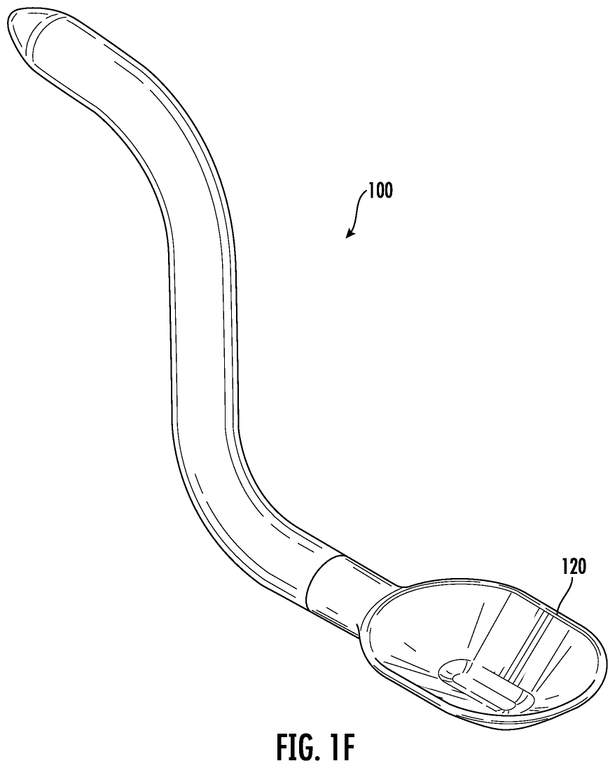

Referring to an embodiment illustrated in FIGS. 1A and 1B, a pelvic floor physical therapy device, generally designated 100, can include a body portion 110 and a handle portion 120. The body portion 110 can be 3D printed, additively manufactured, or developed by any other suitable manufacturing method. In some embodiments, the body portion 110 is formed of a substantially rigid material (e.g., plastic) having a desired shape. In some embodiments, the body portion 110 has a substantially circular cross-sectional shape, with a length of between about 5 inches and about 24 inches (e.g., about 10 inches in some embodiments) and a circumference of up to about 2 inches (e.g., about 0.787 inches in some embodiments). Those having ordinary skill in the art will recognize, however, that the body portion 110 can be produced to have any of a variety of other dimensions based on the requirements of the PT treatment.

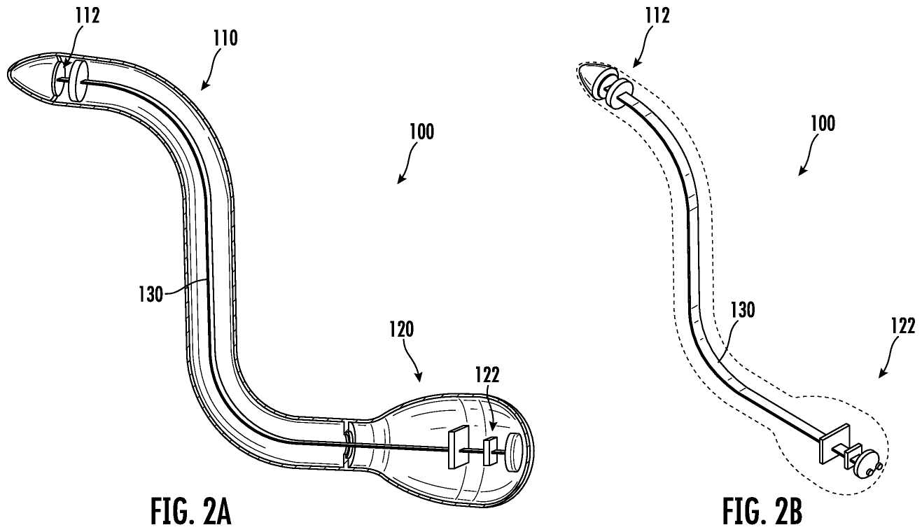

In some embodiments, the body portion 110 has a curved shape that is configured to assist user leverage. As shown in FIGS. 1A and 2B, for example, the body portion 110 can have an S-shaped or substantially S-shaped body. In some embodiments, the handle portion 120 is designed to be easily gripped by a user. In the embodiment shown in FIGS. 1A and 1B, for example, the handle portion 120 has a bulb-like shape that is configured to be griped at any of a variety of angles, although those having ordinary skill in the art will recognize that any of a variety of handle shapes can be used to allow for ergonomic use of the device 100. As shown in FIGS. 1C through 1F, for example, various examples of shapes of handle 112 can include a straight handle (FIG. 1C), a ring-shaped handle (FIG. 1D), a tee-shaped handle (FIG. 1E), and a shoehorn-shaped handle (FIG. 1F). Those having ordinary skill in the art will recognize, however, that any of a variety of other shapes and configurations can be implemented. In any configuration, the handle 112 can be configured to be firmly grasped by the user such that they can comfortably and easily guide the device to the correct spot and then apply the leverage/pressure needed to treat the muscle.

In some embodiments, the device 100 can comprise a coating or covering over an exterior of the device, optionally covering at least a portion of an exterior part of the body portion 110, or at least covering portions of the body portion 110 to be placed in the vagina during therapy. Such a covering could be permanently affixed, or alternatively could be removable. By way of example and not limitation, such a coating can include a biocompatible elastomer material (e.g., silicone) that can be easily disinfected and can be stamped with branding information.

Referring to FIGS. 2A and 2B, the body portion 110 further includes a probe end 112 that includes one or more input and/or feedback elements. In addition, in some embodiments, the handle portion 120 includes an internal component array 122 that is connected to the one or more input and/or feedback elements in the probe end 112 of the body portion 110, such as through a connection cable 130 positioned within the body portion 110 and/or the handle portion 120. Some examples of the input elements can include input sensors that capture pressure/force, temperature, and/or orientation. Pressure/force can be detected using a single sensor or an array of multiple sensors. Temperature can be measured using a thermistor or similar device. Orientation can be detected by a gyroscope, accelerometer, or magnetometer.

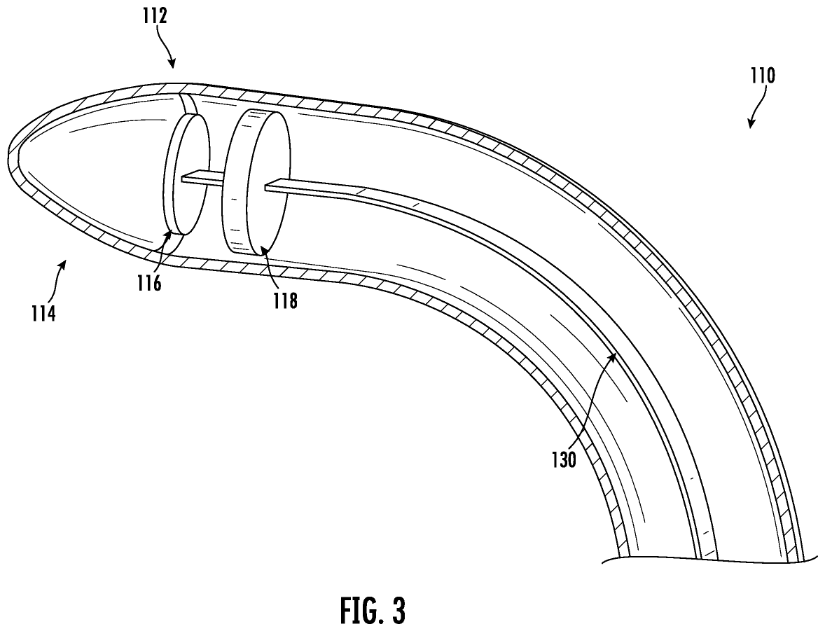

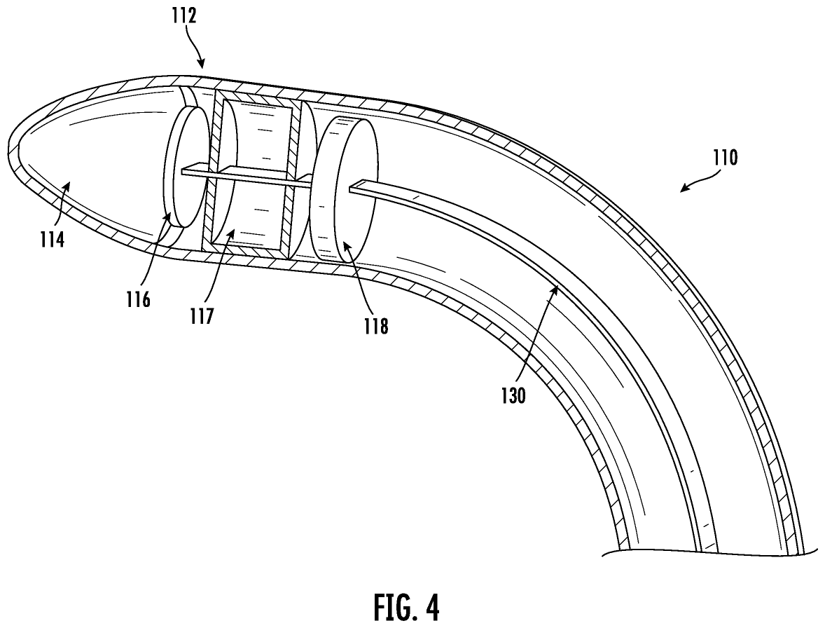

Referring to FIGS. 3 and 4, the probe end 112 of the body portion 110 can include pressure-sensing and vibration features. In an embodiment illustrated in FIG. 3, the probe end 112 includes a balloon tip 114 arranged at an end of the body portion 110 and a pressure sensor 116 in communication with the balloon tip 114. In this configuration, force applied to the balloon tip 114 can be registered by the pressure sensor 116. In some embodiments, the pressure sensor 116 is configured to measure applied pressures of between about 0.0 kg/cm² and about 5.0 kg/cm² with 25 g/cm² resolution. Those having ordinary skill in the art will recognize that any of a variety of other pressure/force sensor configurations can likewise be used, including but not limited to a force-sensitive resistor or a 3-axis capacitive force sensor.

Alternatively or in addition, as illustrated in FIG. 4, the probe end 112 can further include one or more circumferential pressure sensors 117 (e.g., 3-axis pressure sensors) within the body portion 110 and/or a capacitive conductor array arranged along the body portion 110, such as in the first 6-7 cm of the body portion 110 from the tip of the probe end 112. In configurations in which the body portion 110 has a substantially S-shape, the circumferential pressure sensor 117 and/or capacitive conductor array can be arranged in a segment of the body portion 110 before the curve of the S-shape. In some embodiments, the circumferential pressure sensor 117 and/or capacitive conductor array can be configured to detect insertion depth (e.g., up to 9.6 cm with 8 mm resolution). In particular, in some embodiments, the circumferential pressure sensor 117 and/or capacitive conductor array can be configured to detect that the body portion 110 has been inserted to a predefined calibration insertion depth (e.g., about 1 cm).

In either configuration, the pressure sensor array within the device 100 can monitor pressure and guide treatment of a target muscle. The pressure measured can be used to assist in directing a user on how and where to administer therapy based on PT goals and patient symptoms. Moreover, in some embodiments, the device can have the ability to measure contractile strength during a physical therapy exercise (e.g., with a kegel), as well as the ability of the pelvic floor to relax with biofeedback. In some embodiments, for example, contractile strength can be measured using the circumferential pressure sensor 117 either alone or in combination with the pressure sensor 116. In some embodiments, the body portion 110 can include two areas at which contractile strength can be measured: a first area (e.g., about 2 cm from the tip of the probe end 112) configured to measure a comparatively distal strength and a second area (e.g., about 2 cm away from the first area) configured to measure a comparatively proximal strength.

Referring again to FIGS. 3 and 4, the probe end 112 can further include a vibrational component 118 (e.g., a vibration motor) and/or other actuators and control and driver circuitry (not shown) for massage and pressure application. In some embodiments, the vibrational component 118 is configured to operate with a vibration frequency between about 8,000 RPM and about 12,000 RPM, and can be adjusted by the user or therapist as needed or prescribed. In addition, the vibrational component 118 can be configured to provide haptic feedback to inform the user they have reached the correct location (i.e., appropriate depth and location of the selected muscle). In some embodiments, a temperature sensor (not shown) can further be included in the probe end 112, such as behind the pressure sensor 116. A processor can be included for data capture, device control, and data transmission. The data from the sensor technology can also transmit wirelessly (e.g., via Bluetooth) to a connected device to give the user feedback on the pressure they are applying in real time as will be discussed in more detail below.

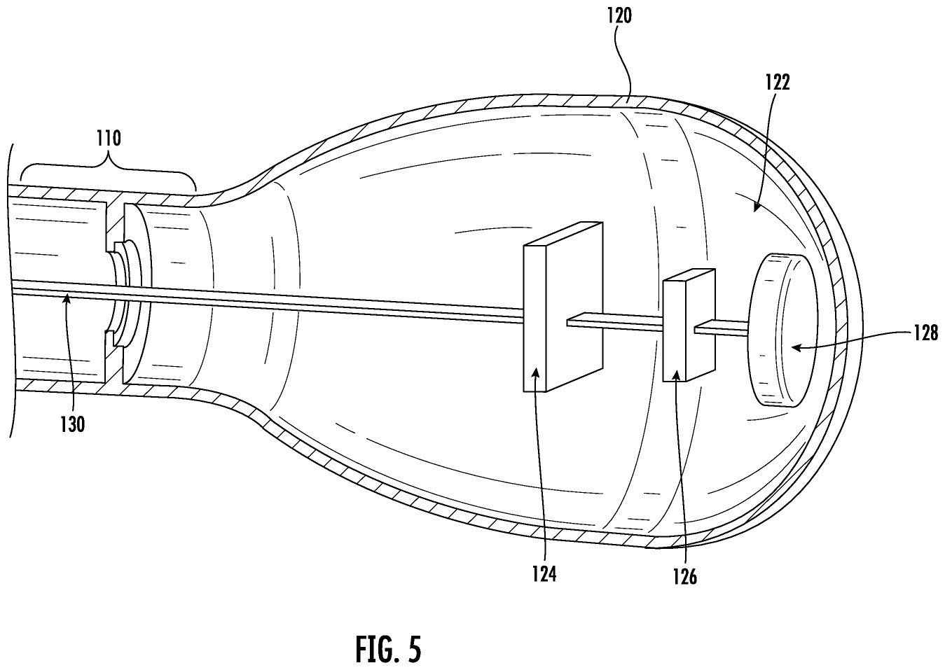

Referring to an embodiment shown in FIG. 5, the internal component array 122 in the handle portion 120 includes a battery 128 and charging circuitry 126 which can be configured to couple to an external base for charging. In this embodiment, the charging circuitry 126 is a wireless charging component that is configured to draw power from an external charging base (not shown) via inductive coupling. An input button 125 can also be used to provide on/off/pairing functionality. An external base station that holds the device 100 can in some embodiments include a rigid plastic case, branding, indicator lights, an inductive coupling charge system, and/or an external AC/DC power supply (not shown). In some embodiments, the device further includes one or more indicator lights to provide information about on/off status, battery charge level, or the like.

Further, in some embodiments, one or both of the probe end 112 of the body portion 110 and/or the internal component array 122 of the handle portion 120 includes one or more orientation sensors (e.g., a gyroscope, accelerometer, or magnetometer) configured to detect the spatial position of the device 100 and/or track the trajectory of the device 100 in real time. In the embodiment shown in FIG. 5, for example, an accelerometer 124 is provided with the internal component array 122, and the position of the probe end 112 can be calculated based on the geometry of the device 100. Those having ordinary skill in the art will recognize, however, that other positions and/or configurations of orientation sensors can be provided in the device 100, including but not limited to positioning an orientation sensor at the probe end 112 (e.g., behind the pressure sensor 116). In some embodiments, the one or more orientation sensors are configured to detect up to 360 degrees of tilt (i.e., pitch and roll) with 0.5 degree resolution.

Regardless of the sensor configuration, the one or more orientation sensors can be spatially calibrated such that the position of the device 100 (e.g., of the probe end 112) is tracked in real time in a model, and its location can be extracted and collected simultaneously. In some embodiments, calibration accuracy can be within about 1 cm, and within about 5 mm in some configurations. The process of spatial calibration can involve identifying the spatial calibration sites and associated metrics, calibrating each image/location against known values, and then applying that calibration to the uncalibrated space. In some embodiments, calibration of the device 100 can be reset at the beginning of each use or therapy/treatment session so that it is appropriately oriented prior to the treatment session. Alternatively, in some embodiments, the therapist/physician/medical professional (referred to collectively as physical therapist) can assist the patient with locating the tender points and directly programming the device to a patients' own pelvis during a scheduled PT appointment.

In embodiments where the vaginal muscles are the points of interest, for example, the device 100 can be oriented such that the probe end 112 contacts the superficial transverse perineal muscle located at the 6 o'clock position immediately upon entry into the vagina. The device 100 can further be oriented such that the probe end 112 contacts the levator ani muscle, the largest component of the pelvic floor, which is located at approximately the 5 and 7 o'clock positions beginning between about 0.5 cm to about 2.0 cm (e.g., about 1 cm) into the vagina just beyond the hymen. In some embodiments, the device 100 can be oriented such that the probe end 112 contacts the obturator internus muscles that lie laterally in the superior inner side of the obturator membrane between about 3 cm and about 5 cm (e.g., approximately 4 cm) into the vagina on each side. This calibration procedure (described further herein) can be performed to map these and/or a plurality of other relevant positions for the pelvic floor PT.

In some embodiments, in each of these locations, the physical therapist can further calibrate the target placements and pressures. In some embodiments, for example, when prompted by the app for each target site (e.g., right pubococcygeus), the physical therapist can position the device 100 in the relevant location with the target amount of pressure and register the location, depth, orientation, and/or pressure data, such as by depressing a button (e.g., either input button 125 or a separate button) or taking a snapshot with a connected device. In addition, in some embodiments, the physical therapist can apply multiple different pressure thresholds (e.g., 1, 2, and 3 kg/cm², in sequential fashion) during the calibration procedure, and the target amount of pressure can be selected at each muscle site for the patient based on participant pressure tolerance (i.e., where pressure is detected without pain).

By calibrating the one or more orientation sensors with these or other physical locations, the constructed “map” of the vagina and muscles to target can be used to guide patients to reach target sites for PT with the recommended depth of insertion, orientation, and/or applied pressure. Alternatively or in addition, the device 100 can be operated in a “discovery mode” that allows users to explore the pelvic floor muscles with the device 100 to find and identify the different muscles of the pelvic floor and identify the problem or painful areas.

Further, in some embodiments, orientation and/or pressure sensor data can be combined with calibration data to provide biofeedback to the user. In some embodiments, an array of sensors is calibrated in software and firmware to map to a designated “global zero” or landmark specific to each user for ease of use. In some embodiments, the device 100 can be calibrated at the vaginal entrance to ensure starting the mapping from a set location prior to initiation of the exercise program. In some embodiments, feedback can be provided in real time via force feedback (e.g., via vibration motor 118). Alternatively or in addition, a module for wireless data transmission (e.g., via Bluetooth) can also be included, and feedback can be provided using a connected device that provides visual cues to help direct therapy.

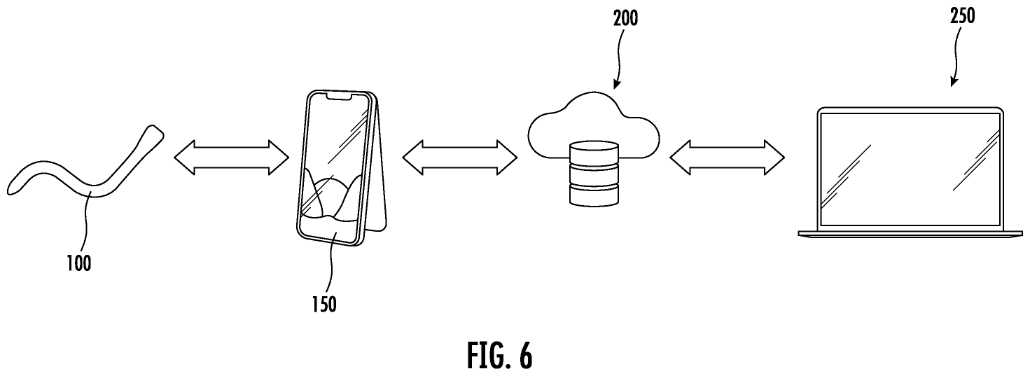

In this regard, in another aspect, the pelvic floor physical therapy device 100 is configured to connect to an app, generally designated 150, which can be provided on a computer or other connected device (e.g., a smartphone) or user interface. Referring to FIG. 6, in some embodiments, the app 150 can be configured to wirelessly and securely transmit and receive data (e.g., via Bluetooth) from the device 100, track progress over time, and/or provide data input to control the device 100. The information collected can further be communicated (e.g., via wireless or cellular communications protocols) to a connected storage device 200 (e.g., a cloud server database).

In some embodiments, the app 150 includes a “front-end” user interface, which can include any of a variety of features, including but not limited to data visualization, controls, tracking, battery monitoring, or therapy direction to assist the user as they perform their pelvic PT floor exercises and track their progress with each session. In some embodiments, the app 150 can provide visual, haptic, and/or auditory feedback regarding the orientation of the device 100 while it's inside the vagina. In some embodiments, spatial positioning of the device 100 (e.g., of probe end 112) is captured and tracked in real time in a model. The movements of the device 100 can be translated to this model, such as by using an iterative closest point-based algorithm. The data can be transmitted wirelessly (e.g., via Bluetooth) to the app 150.

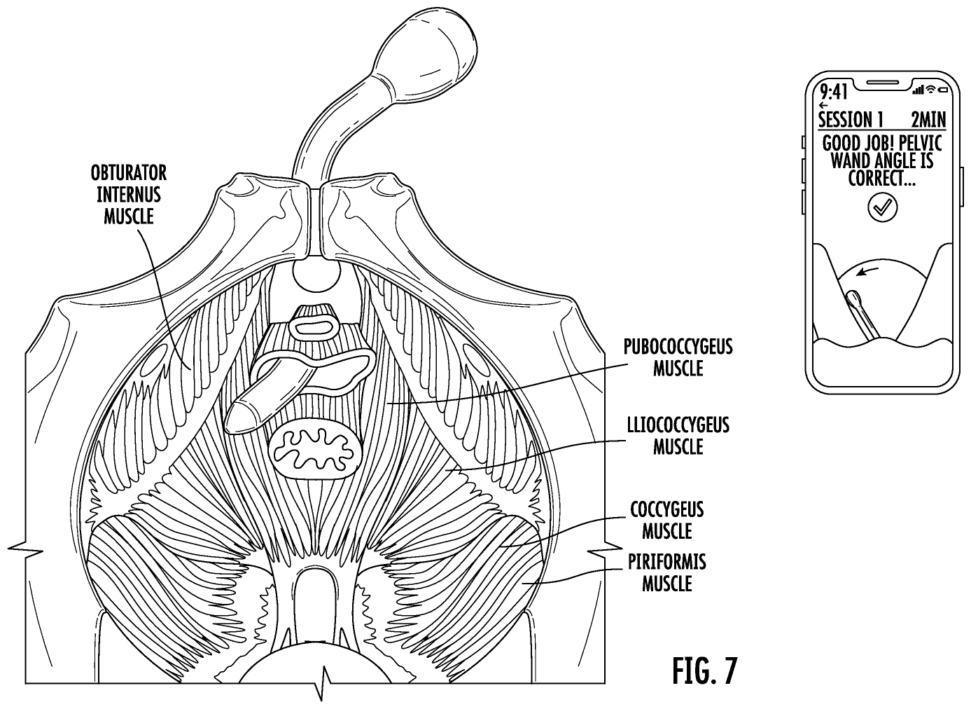

In this way, the app 150 can provide directions to guide the user to adjust the position of the device 100 to be in a desired orientation and/or to notify the user if it's in the wrong place, interpret pressure measurements taken from the built-in sensors, and direct the user to adjust the pressure they are applying in real-time. In some embodiments, for example, the app 150 will sequentially highlight target muscles and provide visual feedback on target insertion depth, tilt, and applied force based on the targets established by the PT. Referring to FIG. 7, for example, the app 150 can be configured to communicate with the device 100 to identify the orientation of the device 100, provide visual and/or auditory feedback to the user to guide the user to reposition the device 100 as necessary to engage the target muscle. In FIG. 7, the device 100 is oriented such that the probe end 112 touches a selected muscle (e.g., for muscle release), and those having ordinary skill in the art will recognize that the user can be guided to change the orientation of the device 100 to engage any of a variety of other muscles of the pelvic floor. The app 150 can also gather pain level data from the patient that is synchronous with the depth, tilt, and pressure data for later analysis.

In some embodiments, the app 150 is further used for the process of spatial calibration of the device 100. Spatial calibration can involve receiving position/orientation data from one or more orientation sensor that is integrated into the device 100 (e.g., in the probe end 112) and tracking the trajectory of the device 100 in real time through a tracking system. As the device 100 is manipulated in the vagina, the position and orientation of the probe end 112 will be communicated to the app 150. In this way, available spatial mapping and pressure sensing technologies can be used to guide the device 100 in space in the vagina. Accurate spatial calibration can thereby improve the image reconstruction and image-guided interventions.

In addition, in some embodiments, the app 150 can ask the user basic introductory questions before use, store each session's data with the purpose of displaying trends over time, ask the user for feedback on their session, provide education on how to use a device 100 for trigger/tender point release, and/or include a positive reinforcement component (i.e., award-based encouragement for compliance and adherence). In some embodiments, the app 150 can record duration of time and amount of pressure applied at a set location (e.g., depth and orientation) in the female pelvis in relation to the pelvic floor muscles, and total self-treatment session time. In addition, in some embodiments, sessions performed and program history can be available in the app 150 and/or in a connected storage device 200 (e.g., a cloud storage device). In some embodiments, the app 150 can provide guidance directly from physical therapists or using AI programming to change or direct care over time. In some embodiments, 2D and 3D ultrasound/MRI or other imaging and functional testing information collected during scheduled physical therapy appointments can be imported into the app 150 to identify myofascial tender points.

In addition, in some embodiments, the app 150 can include user profile information, set interval symptom intake (e.g., daily/weekly symptom-based pain reporting, quality of life measures, activities of daily living), ancillary prescribed body therapy exercise programs (e.g., mindfulness, relaxation, breathing exercises, stretching, yoga), external pelvic exercises programs, internal pelvic exercise programs, and disease-based programming (e.g., information regarding overall high tone pelvic floor dysfunction, vulvodynia, deep dyspareunia, endometriosis-associated pain, bladder pain syndrome, outlet-obstruction constipation, levator ani myalgia, obturator internus myalgia). In some embodiments, the app 150 can further provide the ability to communicate with other appropriate pelvic floor education platforms/forums.

By combining the pelvic floor physical therapy device 100 with an associated app 150, patients with pelvic floor dysfunction can initiate pelvic floor physical therapy either at-home or in a clinical setting under a physical therapist's supervision, which can thereby help improve the accessibility of pelvic floor physical therapy and remedy the problems associated with the prevalent disease state of pelvic floor dysfunction. For example, the present system can be used in conjunction with conventional PFPT therapies as part of a full in-person or hybrid care practice that includes virtual and in-person visits. Alternatively, in some embodiments, the present system can be used as a tool for fully virtual PFPT care guided by a provider remotely. In other embodiments, the present system can be used completely independently (i.e., not under the guidance of a care provider), with the user being guided in the use of the device 100 through exercises provided by the app 150.

In any application, this targeted treatment can allow the user's physical therapy exercises to be more effective, leading to improved clinical outcomes. Additionally, the programming can feature a variety of settings designed for different experiences, such as modes to improve defecatory function (e.g., perineal application for outlet dysfunction) or to focus on introital muscles prior to vaginal penetration.

In some embodiments, the app 150 further includes a “back-end” component that can provide data processing (e.g., cloud-linked), orientation calculation, notification handling, EMR integration, data encryption and security handling. In particular, in some embodiments, the app can include a “mapping” of the pelvic floor muscles and highlight or visualize to a user when a certain set of muscles is being treated. In this way, coordinated operation of the device 100 and the app 150 can be able to identify poorly- or well-coordinated muscles along the length of the vagina and identify potential treatment targets.

In another aspect, the pelvic floor physical therapy device 100 can be used with a software interface to provide communication between the user and a medical professional overseeing the physical therapy. In this way, the therapeutic process can be objectified and clarified in order to improve the accessibility and affordability of pelvic floor physical therapy. Referring again to the embodiment illustrated in FIG. 6, a physical therapy professional interface 250 (e.g., an application on a remote computer) can communicate (e.g., via wired, wireless, or cellular communications protocols) with the storage device 200 that previously received data from the app 150. In this arrangement, data collected from operation of the pelvic floor physical therapy device 100 and/or through communication with the app 150 can be integrated with an existing secure electronic medical record (EMR) for use by providers for prescribing therapy regimens and/or for sending notifications to remind users to complete their exercises.

The connectivity can also allow telemedicine PT visits to be performed in real time with a physical therapist receiving live data and directing patient use via a virtual visit or at set intervals. For example, in some embodiments, the patient can log into the cloud system, receive a treatment program from their physical therapist, and perform the therapeutic exercises independently at home.

Further, in some embodiments, de-identified user data can be collected over time to improve cloud-based algorithms for the identification of certain treatment programs, associate the data with disease phenotypes, and/or to inform future pelvic floor programming that may ultimately allow users to operate the device 100 and app 150 independent of a provider. These features can improve access to care for all patients with PFD, including those who do not have access to physical therapy.

The subject matter disclosed herein can be implemented in or with software in combination with hardware and/or firmware. For example, the subject matter described herein can be implemented in software executed by a processor or processing unit. In one exemplary implementation, the subject matter described herein can be implemented using a computer readable medium having stored thereon computer executable instructions that when executed by a processor of a computer control the computer to perform steps. Exemplary computer readable mediums suitable for implementing the subject matter described herein include non-transitory devices, such as disk memory devices, chip memory devices, programmable logic devices, and application specific integrated circuits. In addition, a computer readable medium that implements the subject matter described herein can be located on a single device or computing platform or can be distributed across multiple devices or computing platforms.

### Definitions

The terminology used herein is for the purpose of describing particular embodiments only and is not intended to be limiting of the presently disclosed subject matter. Although the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “symptom”, as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

What is claimed is:

1. A pelvic floor physical therapy device comprising:
a handle portion configured to be gripped by a user;
a body portion extending from the handle portion;
one or more sensors integrated within the body portion; and
wherein the one or more sensors are configured to collect sensor data regarding a position of the pelvic floor physical therapy device with respect to a pelvic floor location or pelvic floor muscle of a user.

2. The device of claim 1, wherein the handle portion comprises a shape selected from the group consisting of straight handle, a bulb shape, a tee-shape, a ring shape, and a shoehorn.

3. The device of any of claim 1 or claim 2, wherein the body portion is substantially S-shaped.

4. The device of claim 1, wherein at least a portion of the body portion is covered with a biocompatible elastomer material.

5. The device of claim 1, wherein the one or more sensors comprises one or more orientation sensors configured to detect spatial positioning of the body portion.

6. The device of any of claim 1 or claim 5, wherein the one or more sensors comprises one or more pressure sensors configured to detect contact pressures of the body portion with respect to the pelvic floor location or pelvic floor muscle of the user.

7. The device of any of claim 1 or claim 5, comprising one or more vibrational components integrated within the body portion, wherein the one or more vibrational components are configured to provide targeted tender point muscle release and vibrational intravaginal therapy and/or haptic feedback to the user.

8. The device of claim 1, comprising a wireless data transmitter configured to transmit the data wirelessly to a connected computer device.

9. A system for guided pelvic floor physical therapy, the system comprising:
a pelvic floor physical therapy device comprising:
a handle portion configured to be gripped by a user;
a body portion extending from the handle portion;
one or more sensors integrated within the body portion; and
a wireless data transmitter; and
a computer device comprising a display, a wireless data receiver, and a non-transitory computer readable medium programmed with a software application comprising executable instructions, the instructions including:
receiving, from the pelvic floor physical therapy device, data from the one or more sensors regarding a position of the pelvic floor physical therapy device with respect to a pelvic floor location or pelvic floor muscle;
comparing the data to a predetermined model of the pelvic floor location or pelvic floor muscle; and
providing visual or auditory feedback regarding the position of the pelvic floor physical therapy device with respect to the pelvic floor location or pelvic floor muscle to guide a physical therapy treatment session.

10. The system of claim 9, wherein the one or more sensors comprises one or more orientation sensors configured to detect spatial positioning of the body portion.

11. The system of claim 10, wherein the software application comprises executable instructions configured to provide spatial feedback to the user for adjusting the position of the pelvic floor physical therapy device with respect to the pelvic floor location or pelvic floor muscle.

12. The system of any of claim 10 or claim 11, wherein the one or more orientation sensors are configured to detect spatial positioning of a probe end of the body portion; and
wherein the software application comprises executable instructions configured to perform an iterative closest point-based optimization algorithm to estimate a spatial transformation that matches two-point sets regarding the spatial positioning of the probe end with respect to the pelvic floor location or pelvic floor muscle.

13. The system of claim 9, wherein the one or more sensors comprises one or more pressure sensors configured to detect contact pressures of the body portion with respect to the pelvic floor location or pelvic floor muscle.

14. The system of claim 13, wherein the software application comprises executable instructions configured to interpret the contact pressures and direct the user to adjust an applied pressure.

15. The system of claim 9, wherein the software application comprises executable instructions configured to:
store the data from the one or more sensors over the physical therapy treatment session; and
transmit the data to a connected storage device that is configured to be accessible by a medical professional overseeing the physical therapy treatment session.

16. The system of any of claim 9 or claim 15, wherein the software application comprises executable instructions configured to receive, from a remote computer, communication from a medical professional overseeing the physical therapy treatment session; and
wherein providing visual or auditory feedback comprises prescribing therapy regimens.

17. The system of claim 9, wherein the pelvic floor physical therapy device comprises one or more vibrational components integrated within the body portion,
wherein the one or more vibrational components are configured to provide targeted tender point muscle release and vibrational intravaginal therapy and/or haptic feedback to the user.

18. The system of claim 9, further comprising a charging or docking station configured to transfer power to the pelvic floor physical therapy device.

19. A method of treating a subject, the method comprising:
positioning a pelvic floor physical therapy device with respect to a pelvic floor location or pelvic floor muscle of a user;
receiving, from the pelvic floor physical therapy device, data from one or more sensors regarding the position of the pelvic floor physical therapy device with respect to the pelvic floor location or pelvic floor muscle;
comparing the data to a predetermined model of the pelvic floor location or pelvic floor muscle; and
providing visual or auditory feedback regarding the position of the pelvic floor physical therapy device with respect to the pelvic floor location or pelvic floor muscle to guide a physical therapy treatment session.

20. The method of claim 19, wherein receiving data from one or more sensors comprises detecting spatial positioning of the pelvic floor physical therapy device.

21. The method of claim 20, wherein providing visual or auditory feedback comprises providing spatial feedback to the user for adjusting the position of the pelvic floor physical therapy device with respect to the pelvic floor location or pelvic floor muscle.

22. The method of any of claim 20 or claim 21, wherein detecting spatial positioning of the pelvic floor physical therapy device comprises detecting spatial positioning of a probe end of the pelvic floor physical therapy device; and
wherein comparing the data comprises performing an iterative closest point-based optimization algorithm to estimate a spatial transformation that matches two-point sets regarding the spatial positioning of the probe end with respect to the pelvic floor location or pelvic floor muscle.

23. The method of claim 19, wherein receiving data from one or more sensors comprises detecting contact pressures of the pelvic floor physical therapy device with respect to the pelvic floor location or pelvic floor muscle of the user.

24. The method of claim 23, wherein detecting contact pressures comprises measuring contractile strength of the pelvic floor muscle of the user.

25. The method of any of claim 23 or claim 24, wherein comparing the data comprises comparing the contact pressures to predetermined target contact pressures; and
wherein providing visual or auditory feedback comprises directing the user to adjust an applied pressure.

26. The method of claim 19, comprising operating one or more vibrational components of the pelvic floor physical therapy device to provide targeted tender point muscle release and vibrational intravaginal therapy and/or haptic feedback to the user.

27. The method of claim 19, wherein positioning a pelvic floor physical therapy device comprises positioning a pelvic floor physical therapy device within a vagina of the user in contact with the pelvic floor to treat pelvic floor dysfunction (PFD) and related conditions.

28. The method of claim 19, wherein positioning a pelvic floor physical therapy device comprises positioning a pelvic floor physical therapy device for rectal application and treating rectal conditions.

29. The method of claim 19, comprising storing the data from the one or more sensors over the physical therapy treatment session; and
transmitting the data to a connected storage device that is configured to be accessible by a physical therapist or physician overseeing the physical therapy treatment.

30. The method of any of claim 19 or claim 29, comprising receiving, from a remote computer, communication from a medical professional overseeing the physical therapy treatment session;
wherein providing visual or auditory feedback comprises prescribing therapy regimens.
